(19) United States
(12) Patent Application Publication
(10) Pub. No.: US 2002/0099414 A1
(43) Pub. Date: Jul. 25, 2002

(54) Method and system for preventing the recurrence of atrial fibrillation by an implantable medical device

(76) Inventors: Xander Evers, Dieren (NL); Johannes S. Van Der Veen, Dieren (NL)

Correspondence Address: Medtronic, Inc., 710 Medtronic Parkway N.E., Minneapolis, MN, 55432, US

(21) Appl. No.: 09/727,460

(22) Filed: Dec. 4, 2000

(51) Int. Cl. A61N 1/362

(52) U.S. Cl. 607/14

(57) ABSTRACT

A method for preventing the recurrence of an atrial fibrillation of a heart is provided. An atrial fibrillation is detected. A set of pacing pulses to increase a rate of the heart to an intervention rate is transmitted to the heart upon the completion of the atrial fibrillation. Finally, the intervention rate is maintained for a predetermined period of time.

### FIELD OF THE INVENTION

The present invention relates to cardial pacing systems and methods, and, more particularly, to cardial pacing systems which provide for a method and system for preventing the occurrence of atrial fibrillation by an implantable medical device.

### BACKGROUND OF THE INVENTION

Atrial fibrillation is a common disease responsible for substantial morbidity and resource consumption. For many patients, the natural history of paroxysmal atrial fibrillation is degeneration to the chronic form of this disease. It was originally thought that the reason for this transition to chronicity was due solely to a progression of the underlying disease process. Unfortunately, for many patients, this process is an idiopathic degeneration and fibrotic replacement of the atrial tissue, for which there is no therapy. It has been demonstrated, however, that paroxysms of atrial fibrillation lead to atrial electrophysiologic changes in both humans and animals, that are thought to promote the persistence of this arrhythmia and that make maintenance of this sinus rhythm more difficult. This is important from a therapeutic standpoint because it justifies aggressive efforts to minimize the frequency and duration of such paroxysms to avoid this progressive self-perpetuating process.

The only available therapy for atrial fibrillation and other cardiac tachyarrhythmias had previously consisted of antiarrhythmiac drugs. Increasingly, non-pharamacologic therapeutic modalities for cardiac arrhythmias have been developed, and in many cases have replaced antiarrhythmiac drugs as first-line therapy. This trend has been motivated by the realization of the potentially lethal side effects of antiarrhythmiac drugs as well as their frequent suboptimal efficacy and tolerance. Furthermore, for many patients and physicians, the concept of a therapeutic modality that is activated only when needed is more appealing than pharmacotherapy, which must be continually administered, regardless of its needs. The success of nonpharmacologic therapeutic modalities for other arrhythmias have led to their application in the treatment of atrial fibrillation; the atrial defibrillator is undergoing clinical trials, and preliminary results of radiofrequency ablation as a potential cure for atrial fibrillation have been presented.

Furthermore, pacing literature has pointed out that atrial pacing may be effective in stabilizing otherwise unstable atria, as well as for the prevention of atrial tachyarrhythmias, for example, “Cardiac Pacing in Special and Complex Situations,” Cardiology Clinics, pages 573-91, 1992; “A New Pacing Algorithm for Suppression of Atrial Fibrillation,” PACE 17, Part II, page 863. Several pacemakers and defibrillators have utilized this principle, including, for example, Vitatron's DPG™ and Selection™, as well as Medtronic's Kappa™.

Modifying the pacing rate for the pacing interval for various purposes has been shown in the past also. See, for example, in defining the atrial-ventricular escape interval supplied to HOCM pacing, as in “Permanent Pacing as Treatment for Hypertropic Cardiomyopathy,” “American Journal of Cardiology,” Volume 68, pages 108-10. Hysteresis has been provided so that the pacer may turn itself off in the presence of naturally conducted depolarizations. For this later category, see Bowers, U.S. Pat. No. 4,030,510, and Sutton, U.S. Pat. No. 5,284,491.

Doctors have recently begun to recognize that dual-chamber pacemakers by themselves seem to reduce the presence of both atrial tachyarrhythmias and atrial fibrillations. See Ishakawa et al., “Preventative Effects of Pacemakers on Paroxysmal Atrial Fibrillation in Patients with Bradycardia-Tachycardia Syndrome,” Artificial Organs, 1994.

Additionally, Hill et al., U.S. Pat. No. 5,403,356 discloses a method and apparatus for pacing an atrium to reduce the incidence of dangerous arrhythmias. In Hill, the invention defines a variable interval following atrial depolarization. This interval is based on the detected rate of depolarization. In response to a sensed depolarization, pacing pulses are delivered to electrodes to increase the pacing of the heart.

Hess et al., U.S. Pat. No. 5,713,929 discloses a pacing algorithm that defines a “faster than indicated” pacing rate during the detection of premature atrial contractions. The invention then reduces the rate after a period of time to a safer rate in the event hat natural depolarizations are not detected.

Finally, Begemann et al., U.S. Pat. No. 5,978,709 discloses a pacemaker system with various improved techniques and methods for preventing and suppressing atrial arrhythmias. Begemann provides for atrial “pace conditioning,” where the patient's normal intrinsic atrial rate is overridden by higher rate pacing whenever a predetermined sequence of intrinsic heartbeats is sensed.

As discussed above, the most pertinent prior art patents are shown in the following table:

TABLE 1
Prior Art Patents.

| Patent No. | Date | Inventor(s) |
|---|---|---|
| 5,978,709 | 11-02-99 | Begemann et al. |
| 5,713,929 | 02-03-98 | Hess et al. |
| 5,403,356 | 04-04-95 | Hill et al. |
| 5,284,491 | 02-08-94 | Sutton |
| 4,030,510 | 06-21-77 | Bowers |

All the patents listed in Table 1 are hereby incorporated by reference herein in their respective entireties. As those of ordinary skill in the art will appreciate readily upon reading the Summary of the Invention, the Detailed Description of the Preferred Embodiments and the claims set forth below, many of the devices and methods disclosed in the patents of Table 1 may be modified advantageously by using the teachings of the present invention.

### SUMMARY OF THE INVENTION

The present invention is therefore directed to providing a method and system for increasing the pace of a mammalian heart by an implantable medical device to prevent recurrence of an atrial fibrillation. Such a system of the present invention overcomes the problems, disadvantages and limitations of the prior art described above, and provides a more efficient and accurate means of increasing the pace of a mammalian heart to prevent recurrence of an atrial fibrillation.

The present invention has certain objects. That is, various embodiments of the present invention provide solutions to one or more problems existing in the prior art respecting the increment of the pace of a mammalian heart to prevent recurrence of an atrial fibrillation. Those problems include, without limitation: the ability to determine when a mammalian heart undergoes an atrial fibrillation, the ability to monitor the heart rate during the atrial fibrillation, the ability to increase the heart rate after the atrial fibrillation, the ability to prevent future recurrences of atrial fibrillation and the ability to avoid symptoms of atrial fibrillation by gradual rate increases and decreases.

In comparison to known techniques for increasing the pace of a mammalian heart to prevent recurrence of an atrial fibrillation, various embodiments of the present invention may provide the following advantages, inter alia, i.e., the detection of an atrial fibrillation, the monitoring of a heart rate during atrial fibrillation, the pacing of the heart with an elevated rate after atrial fibrillation, the reduction of the elevated rate after a predetermined period of time, the prevention of recurrences of paroxysmal atrial fibrillations and other atrial tachyarrhythmias, the achievement of the present invention by an independently programmable intervention rate, gradual escape rate decreases and increases and a smooth transition of the escape rate during the occurrence of an atrial tachyarrhythmia.

Some of the embodiments of the present invention include one or more of the following features: an implantable medical device including at least one sensing lead, at least one pacing lead, a microprocessor and an input/output circuit including a digital controller/timer circuit, an output amplifier, a sense amplifier, a peak sense and threshold measurement device, a comparator and an electrogram amplifier.

Furthermore, in accordance with the present invention, a method for preventing the recurrence of an atrial fibrillation of a heart is provided. An atrial fibrillation is detected. A set of pacing pulses to increase a rate of the heart to an intervention rate is transmitted to the heart upon the completion of the atrial fibrillation. Finally, the intervention rate is maintained for a predetermined period of time.

### BRIEF DESCRIPTION OF THE DRAWINGS

The above, and other objects, advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic view of an embodiment of an implantable medical device, made in accordance with the present invention;

FIG. 2 is another view of the implantable medical device of FIG. 1, made in accordance with the present invention;

FIG. 3 shows a block diagram illustrating the components of the implantable medical device of FIG. 1, made in accordance with the present invention;

FIG. 4 illustrates another embodiment of an implantable medical device, made in accordance with the present invention;

FIG. 5 illustrates a block diagram of the embodiment of FIG. 4, made in accordance with the present invention;

FIG. 6 illustrates a flow chart of a routine of one embodiment of a method of preventing the recurrence of an atrial fibrillation of a heart; and

FIG. 7 illustrates a graphical representation of a routine of one embodiment of a method of preventing the recurrence of an atrial fibrillation of a heart.

### DETAILED DESCRIPTION OF THE PRESENTLY PREFERRED EMBODIMENTS

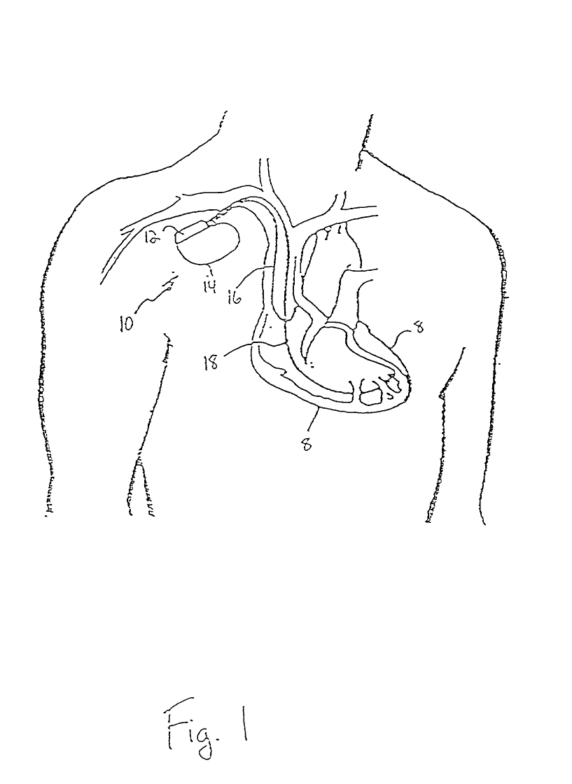

FIG. 1 is a simplified schematic view of one embodiment of implantable medical device (“IMD”) 10 of the present invention. IMD 10 shown in FIG. 1 is a pacemaker comprising at least one of pacing and sensing leads 16 and 18 attached to hermetically sealed enclosure 14 and implanted near human or mammalian heart 8. Pacing and sensing leads 16 and 18 sense electrical signals attendant to the depolarization and re-polarization of the heart 8, and further provide pacing pulses for causing depolarization of cardiac tissue in the vicinity of the distal ends thereof. Leads 16 and 18 may have unipolar or bipolar electrodes disposed thereon, as is well known in the art. Examples of IMD 10 include implantable cardiac pacemakers disclosed in U.S. Pat. No. 5,158,078 to Bennett et al., U.S. Pat. No. 5,312,453 to Shelton et al. or U.S. Pat. No. 5,144,949 to Olson, all hereby incorporated by reference herein, each in its respective entirety.

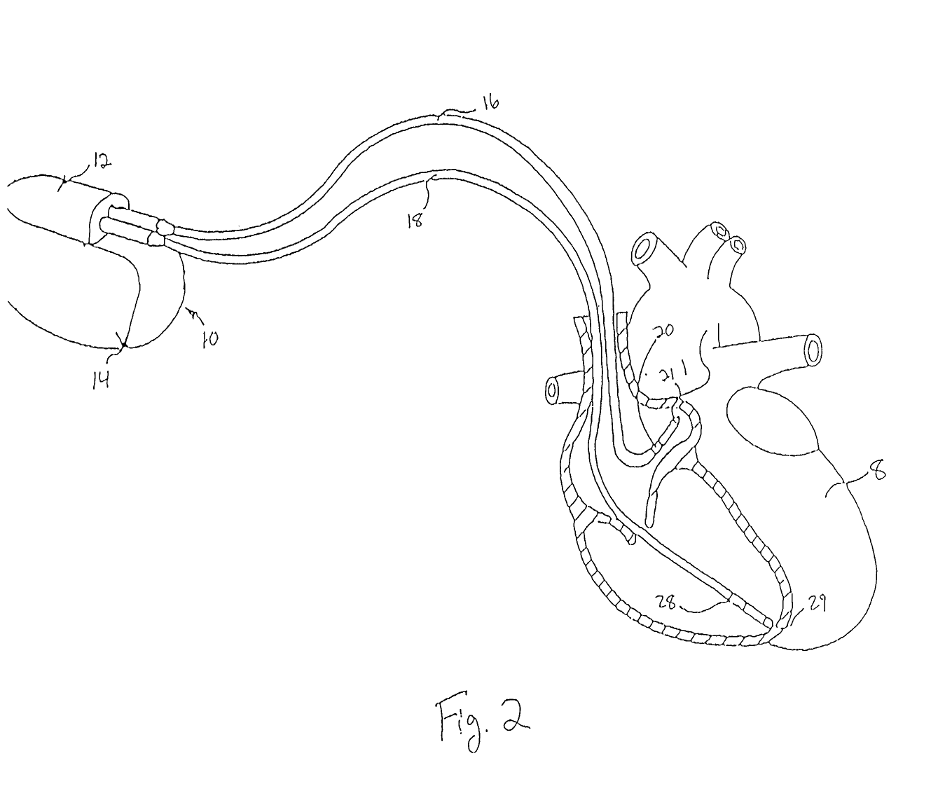

FIG. 2 shows connector module 12 and hermetically sealed enclosure 14 of IMD 10 located in and near human or mammalian heart 8. Atrial and ventricular pacing leads 16 and 18 extend from connector header module 12 to the right atrium and ventricle, respectively, of heart 8. Atrial electrodes 20 and 21 disposed at the distal end of atrial pacing lead 16 are located in the right atrium. Ventricular electrodes 28 and 29 at the distal end of ventricular pacing lead 18 are located in the right ventricle.

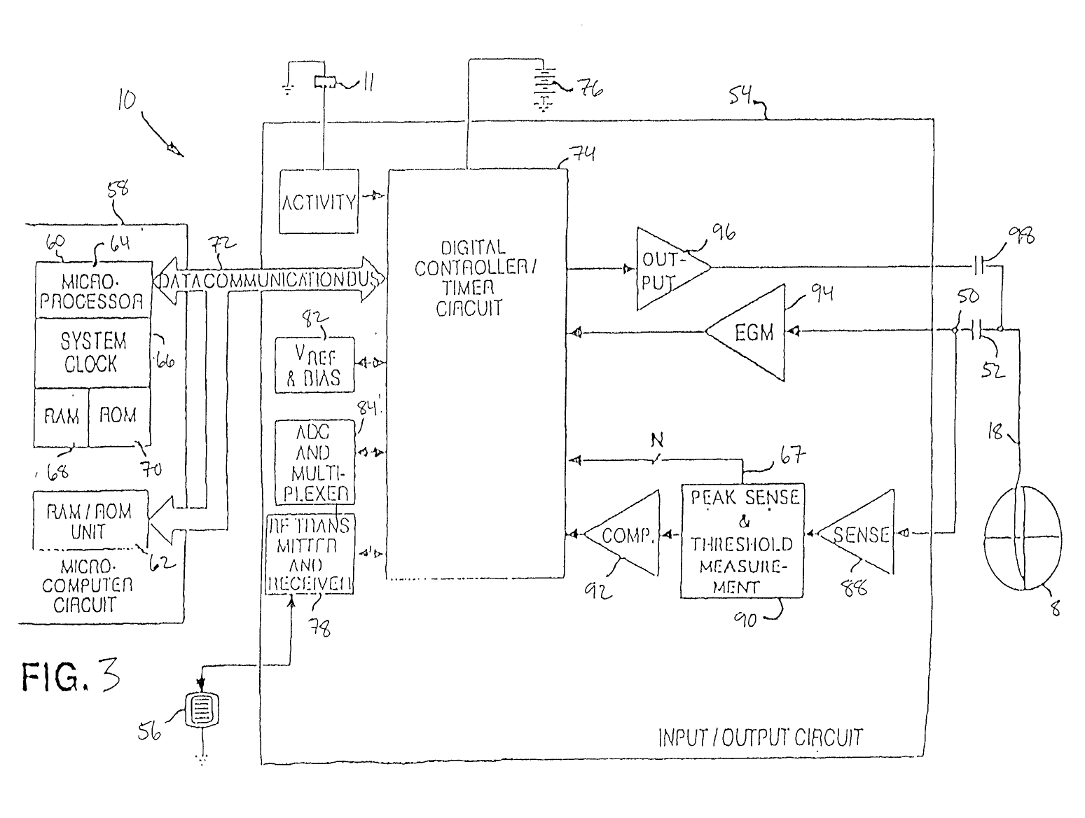

FIG. 3 shows a block diagram illustrating the constituent components of IMD 10 in accordance with one embodiment of the present invention, where IMD 10 is pacemaker having a microprocessor-based architecture. IMD 10 is shown as including activity sensor or accelerometer 11, which is preferably a piezoceramic accelerometer bonded to a hybrid circuit located inside enclosure 14. Activity sensor 11 typically (although not necessarily) provides a sensor output that varies as a function of a measured parameter relating to a patient's metabolic requirements. Forthe sake of convenience, IMD 10 in FIG. 3 is shown with lead 18 only connected thereto; similar circuitry and connections not explicitly shown in FIG. 3 apply to lead 16.

IMD 10 in FIG. 3 is most preferably programmable by means of an external programming unit (not shown in the Figures). One such programmer is the commercially available Medtronic Model 9790 programmer, which is microprocessor-based and provides a series of encoded signals to IMD 10, typically through a programming head which transmits or telemeters radio-frequency (RF) encoded signals to IMD 10. Such a telemetry system is described in U.S. Pat. No. 5,312,453 to Wyborny et al., hereby incorporated by reference herein in its entirety. The programming methodology disclosed in Wyborny et al.'s '453 patent is identified herein for illustrative purposes only. Any of a number of suitable programming and telemetry methodologies known in the art may be employed so long as the desired information is transmitted to and from the pacemaker.

As shown in FIG. 3, lead 18 is coupled to node 50 in IMD 10 through input capacitor 52. Activity sensor or accelerometer 11 is most preferably attached to a hybrid circuit located inside hermetically sealed enclosure 14 of IMD 10. The output signal provided by activity sensor 11 is coupled to input/output circuit 54. Input/output circuit 54 contains analog circuits for interfacing to heart 8, activity sensor 11, antenna 56 and circuits for the application of stimulating pulses to heart 8. The rate of heart 8 is controlled by software-implemented algorithms stored microcomputer circuit 58.

Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66 and on-board RAM 68 and ROM 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components.

Electrical components shown in FIG. 3 are powered by an appropriate implantable battery power source 76 in accordance with common practice in the art. For the sake of clarity, the coupling of battery power to the various components of IMD 10 is not shown in the Figures. Antenna 56 is connected to input/output circuit 54 to permit uplink/downlink telemetry through RF transmitter and receiver telemetry unit 78. By way of example, telemetry unit 78 may correspond to that disclosed in U.S. Pat. No. 4,566,063 issued to Thompson et al., hereby incorporated by reference herein in its entirety, or to that disclosed in the above-referenced '453 patent to Wyborny et al. It is generally preferred that the particular programming and telemetry scheme selected permit the entry and storage of cardiac rate-response parameters. The specific embodiments of antenna 56, input/output circuit 54 and telemetry unit 78 presented herein are shown for illustrative purposes only, and are not intended to limit the scope of the present invention.

Continuing to refer to FIG. 3, VREF and Bias circuit 82 most preferably generates stable voltage reference and bias currents for analog circuits included in input/output circuit 54. Analog-to-digital converter (ADC) and multiplexer unit 84 digitizes analog signals and voltages to provide “real-time” telemetry intracardiac signals and battery end-of-life (EOL) replacement functions. Operating commands for controlling the timing of IMD 10 are coupled by data bus 72 to digital controller/timer circuit 74, where digital timers and counters establish the overall escape interval of the IMD 10 as well as various refractory, blanking and other timing windows for controlling the operation of peripheral components disposed within input/output circuit 54.

Digital controller/timer circuit 74 is preferably coupled to sensing circuitry, including sense amplifier 88, peak sense and threshold measurement unit 90 and comparator/threshold detector 92. Circuit 74 is further preferably coupled to electrogram (EGM) amplifier 94 for receiving amplified and processed signals sensed by lead 18. Sense amplifier 88 amplifies sensed electrical cardiac signals and provides an amplified signal to peak sense and threshold measurement circuitry 90, which in turn provides an indication of peak sensed voltages and measured sense amplifier threshold voltages on multiple conductor signal path 67 to digital controller/timer circuit 74. An amplified sense amplifier signal is then provided to comparator/threshold detector 92. By way of example, sense amplifier 88 may correspond to that disclosed in U.S. Pat. No. 4,379,459 to Stein, hereby incorporated by reference herein in its entirety.

The electrogram signal provided by EGM amplifier 94 is employed when IMD 10 is being interrogated by an external programmer to transmit a representation of a cardiac analog electrogram. See for example, U.S. Pat. No. 4,556,063 to Thompson et al., hereby incorporated by reference herein in its entirety. Output pulse generator 96 provides pacing stimuli to patient's heart 8 through coupling capacitor 98 in response to a pacing trigger signal provided by digital controller/timer circuit 74 each time the escape interval times out, an externally transmitted pacing command is received or in response to other stored commands as is well known in the pacing art. By way of example, output amplifier 96 may correspond generally to an output amplifier disclosed in U.S. Pat. No. 4,476,868 to Thompson, hereby incorporated by reference herein in its entirety.

The specific embodiments of input amplifier 88, output amplifier 96 and EGM amplifier 94 identified herein are presented for illustrative purposes only, and are not intended to be limiting in respect of the scope of the present invention. The specific embodiments of such circuits may not be critical to practicing some embodiments of the present invention so long as they provide means for generating a stimulating pulse and are capable of providing signals indicative of natural or stimulated contractions of heart 8.

In some preferred embodiments of the present invention, IMD 10 may operate in various non-rate-responsive modes, including, but not limited to, AAI, AAIR, AAT, AATR, DDD, DDI, and DDIR modes. Some embodiments of the present invention are capable of operating in both non-rate-responsive and rate responsive modes. Moreover, in various embodiments of the present invention IMD 10 may be programmably configured to operate so that it varies the rate at which it delivers stimulating pulses to heart 8 only in response to one or more selected sensor outputs being generated. Numerous pacemaker features and functions not explicitly mentioned herein may be incorporated into IMD 10 while remaining within the scope of the present invention.

The present invention is not limited in scope to single-sensor or dual-sensor pacemakers, and is not limited to IMD's comprising activity or pressure sensors only. Nor is the present invention limited in scope to single-chamber pacemakers, single-chamber leads for pacemakers or single-sensor or dual-sensor leads for pacemakers. Thus, various embodiments of the present invention may be practiced in conjunction with more than two leads or with multiple-chamber pacemakers, for example. At least some embodiments of the present invention may be applied equally well in the contexts of single-, dual-, triple- or quadruple-chamber pacemakers or other types of IMD's. See for example, U.S. Pat. No. 5,800,465 to Thompson et al., hereby incorporated by reference herein in its entirety, as are all U.S. Patents referenced therein.

IMD 10 may also be a pacemaker-cardioverter-defibrillator (“PCD”) corresponding to any of numerous commercially available implantable PCD's. Various embodiments of the present invention may be practiced in conjunction with PCD's such as those disclosed in U.S. Pat. No. 5,545,186 to Olson et al., U.S. Pat. No. 5,354,316 to Keimel, U.S. Pat. No. 5,314,430 to Bardy, U.S. Pat. No. 5,131,388 to Pless and U.S. Pat. No. 4,821,723 to Baker et al., all hereby incorporated by reference herein, each in its respective entirety.

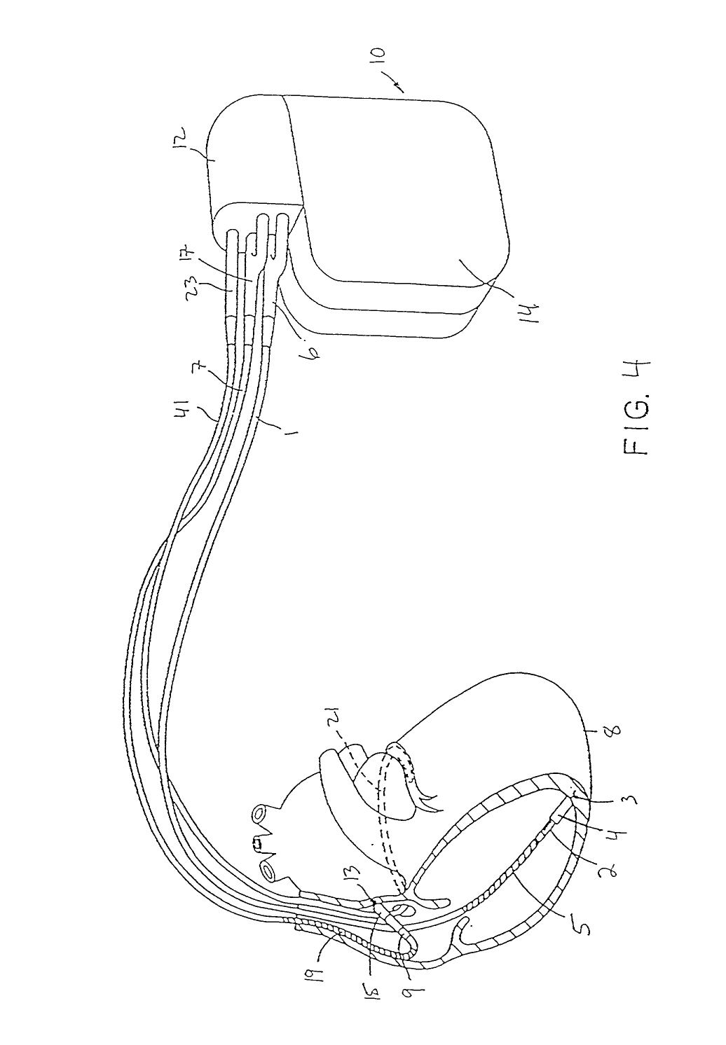

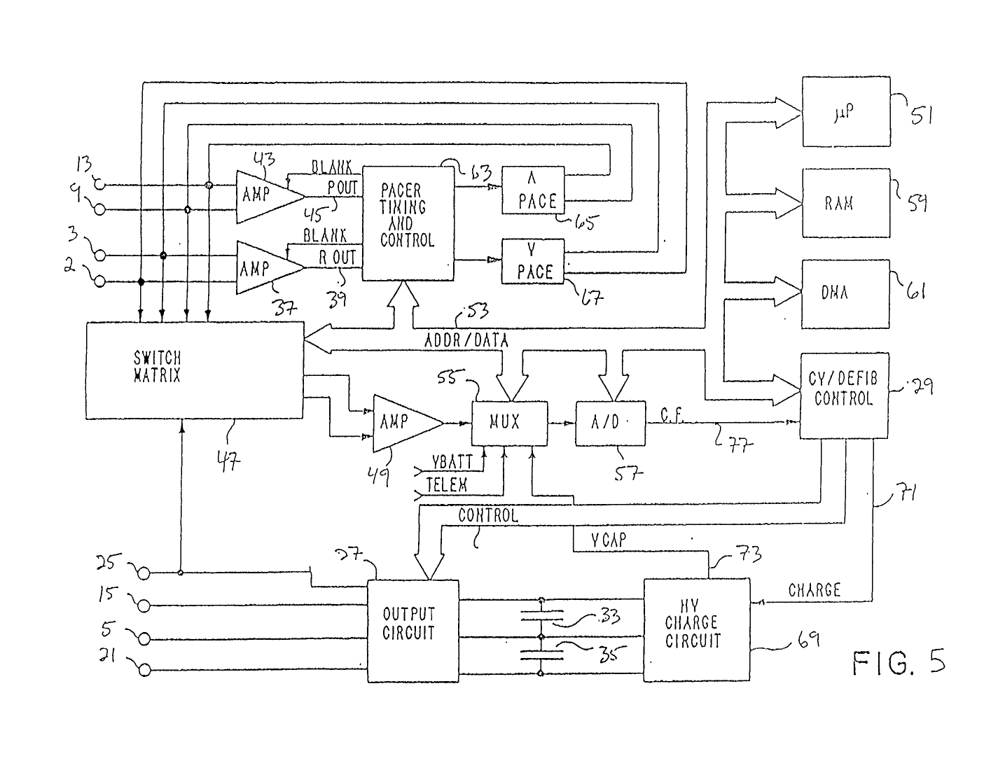

FIGS. 4 and 5 illustrate one embodiment of IMD 10 and a corresponding lead set of the present invention, where IMD 10 is a PCD. In FIG. 4, the ventricular lead takes the form of leads disclosed in U.S. Pat. Nos. 5,099,838 and 5,314,430 to Bardy, and includes an elongated insulative lead body 1 carrying three concentric coiled conductors separated from one another by tubular insulative sheaths. Located adjacent the distal end of lead 1 are ring electrode 2, extendable helix electrode 3 mounted retractably within insulative electrode head 4 and elongated coil electrode 5. Each of the electrodes is coupled to one of the coiled conductors within lead body 1. Electrodes 2 and 3 are employed for cardiac pacing and for sensing ventricular depolarizations. At the proximal end of the lead is bifurcated connector 6 which carries three electrical connectors, each coupled to one of the coiled conductors. Defibrillation electrode 5 may be fabricated from platinum, platinum alloy or other materials known to be usable in implantable defibrillation electrodes and may be about 5 cm in length.

The atrial/SVC lead shown in FIG. 4 includes elongated insulative lead body 7 carrying three concentric coiled conductors separated from one another by tubular insulative sheaths corresponding to the structure of the ventricular lead. Located adjacent the J-shaped distal end of the lead are ring electrode 9 and extendable helix electrode 13 mounted retractably within an insulative electrode head 15. Each of the electrodes is coupled to one of the coiled conductors within lead body 7. Electrodes 13 and 9 are employed for atrial pacing and for sensing atrial depolarizations. Elongated coil electrode 19 is provided proximal to electrode 9 and coupled to the third conductor within lead body 7. Electrode 19 preferably is 10 cm in length or greater and is configured to extend from the SVC toward the tricuspid valve. In one embodiment of the present invention, approximately 5 cm of the right atrium/SVC electrode is located in the right atrium with the remaining 5 cm located in the SVC. At the proximal end of the lead is bifurcated connector 17 carrying three electrical connectors, each coupled to one of the coiled conductors.

The coronary sinus lead shown in FIG. 4 assumes the form of a coronary sinus lead disclosed in the above cited '838 patent issued to Bardy, and includes elongated insulative lead body 41 carrying one coiled conductor coupled to an elongated coiled defibrillation electrode 21. Electrode 21, illustrated in broken outline in FIG. 4, is located within the coronary sinus and great vein of the heart. At the proximal end of the lead is connector plug 23 carrying an electrical connector coupled to the coiled conductor. The coronary sinus/great vein electrode 41 may be about 5 cm in length.

Implantable PCD 10 is shown in FIG. 4 in combination with leads 1, 7 and 41, and lead connector assemblies 23, 17 and 6 inserted into connector block 12. Optionally, insulation of the outward facing portion of housing 14 of PCD 10 may be provided using a plastic coating such as parylene or silicone rubber, as is employed in some unipolar cardiac pacemakers. The outward facing portion, however, may be left uninsulated or some other division between insulated and uninsulated portions may be employed. The uninsulated portion of housing 14 serves as a subcutaneous defibrillation electrode to defibrillate either the atria or ventricles. Lead configurations other that those shown in FIG. 4 may be practiced in conjunction with the present invention, such as those shown in U.S. Pat. No. 5,690,686 to Min et al., hereby incorporated by reference herein in its entirety.

FIG. 5 is a functional schematic diagram of one embodiment of implantable PCD 10 of the present invention. This diagram should be taken as exemplary of the type of device in which various embodiments of the present invention may be embodied, and not as limiting, as it is believed that the invention may be practiced in a wide variety of device implementations, including cardioverter and defibrillators which do not provide anti-tachycardia pacing therapies.

IMD 10 is provided with an electrode system. If the electrode configuration of FIG. 4 is employed, the correspondence to the illustrated electrodes is as follows. Electrode 25 in FIG. 5 includes the uninsulated portion of the housing of PCD 10. Electrodes 25, 15, 21 and 5 are coupled to high voltage output circuit 27, which includes high voltage switches controlled by CV/defib control logic 29 via control bus 31. Switches disposed within circuit 27 determine which electrodes are employed and which electrodes are coupled to the positive and negative terminals of the capacitor bank (which includes capacitors 33 and 35) during delivery of defibrillation pulses.

Electrodes 2 and 3 are located on or in the ventricle and are coupled to the R-wave amplifier 37, which preferably takes the form of an automatic gain controlled amplifier providing an adjustable sensing threshold as a function of the measured R-wave amplitude. A signal is generated on R-out line 39 whenever the signal sensed between electrodes 2 and 3 exceeds the present sensing threshold.

Electrodes 9 and 13 are located on or in the atrium and are coupled to the P-wave amplifier 43, which preferably also takes the form of an automatic gain controlled amplifier providing an adjustable sensing threshold as a function of the measured P-wave amplitude. A signal is generated on P-out line 45 whenever the signal sensed between electrodes 9 and 13 exceeds the present sensing threshold. The general operation of R-wave and P-wave amplifiers 37 and 43 may correspond to that disclosed in U.S. Pat. No. 5,117,824, by Keimel et al., issued Jun. 2, 1992, for “An Apparatus for Monitoring Electrical Physiologic Signals,” hereby incorporated by reference herein in its entirety.

Switch matrix 47 is used to select which of the available electrodes are coupled to wide band (0.5-200 Hz) amplifier 49 for use in digital signal analysis. Selection of electrodes is controlled by the microprocessor 51 via data/address bus 53, which selections may be varied as desired. Signals from the electrodes selected for coupling to bandpass amplifier 49 are provided to multiplexer 55, and thereafter converted to multi-bit digital signals by A/D converter 57, for storage in random access memory 59 under control of direct memory access circuit 61. Microprocessor 51 may employ digital signal analysis techniques to characterize the digitized signals stored in random access memory 59 to recognize and classify the patient's heart rhythm employing any of the numerous signal processing methodologies known to the art.

The remainder of the circuitry is dedicated to the provision of cardiac pacing, cardioversion and defibrillation therapies, and, for purposes of the present invention may correspond to circuitry known to those skilled in the art. The following exemplary apparatus is disclosed for accomplishing pacing, cardioversion and defibrillation functions. Pacer timing/control circuitry 63 preferably includes programmable digital counters which control the basic time intervals associated with DDD, WI, DVI, VDD, AAI, DDI and other modes of single and dual chamber pacing well known to the art. Circuitry 63 also preferably controls escape intervals associated with anti-tachyarrhythmia pacing in both the atrium and the ventricle, employing any anti-tachyarrhythmia pacing therapies known to the art.

Intervals defined by pacing circuitry 63 include atrial and ventricular pacing escape intervals, the refractory periods during which sensed P-waves and R-waves are ineffective to restart timing of the escape intervals and the pulse widths of the pacing pulses. The durations of these intervals are determined by microprocessor 51, in response to stored data in memory 59 and are communicated to pacing circuitry 63 via address/data bus 53. Pacer circuitry 63 also determines the amplitude of the cardiac pacing pulses under control of microprocessor 51.

During pacing, escape interval counters within pacer timing/control circuitry 63 are reset upon sensing of R-waves and P-waves as indicated by a signals on lines 39 and 45, and in accordance with the selected mode of pacing on time-out trigger generation of pacing pulses by pacer output circuitry 65 and 67, which are coupled to electrodes 9, 13, 2 and 3. Escape interval counters are also reset on generation of pacing pulses and thereby control the basic timing of cardiac pacing functions, including anti-tachyarrhythmia pacing. The durations of the intervals defined by escape interval timers are determined by microprocessor 51 via data/address bus 53. The value of the count present in the escape interval counters when reset by sensed R-waves and P-waves may be used to measure the durations of R-R intervals, P-P intervals, P-R intervals and R-P intervals, which measurements are stored in memory 59 and used to detect the presence of tachyarrhythmias.

Microprocessor 51 most preferably operates as an interrupt driven device, and is responsive to interrupts from pacer timing/control circuitry 63 corresponding to the occurrence sensed P-waves and R-waves and corresponding to the generation of cardiac pacing pulses. Those interrupts are provided via data/address bus 53. Any necessary mathematical calculations to be performed by microprocessor 51 and any updating of the values or intervals controlled by pacer timing/control circuitry 63 take place following such interrupts.

Detection of atrial or ventricular tachyarrhythmias, as employed in the present invention, may correspond to tachyarrhythmia detection algorithms known in the art. For example, the presence of an atrial or ventricular tachyarrhythmia may be confirmed by detecting a sustained series of short R-R or P-P intervals of an average rate indicative of tachyarrhythmia or an unbroken series of short R-R or P-P intervals. The suddenness of onset of the detected high rates, the stability of the high rates, and a number of other factors known in the art may also be measured at this time. Appropriate ventricular tachyarrhythmia detection methodologies measuring such factors are described in U.S. Pat. No.4,726,380 issued to Vollmann, U.S. Pat. No. 4,880,005 issued to Pless et al. and U.S. Pat. No. 4,830,006 issued to Haluska et al., all incorporated by reference herein, each in its respective entirety. An additional set of tachycardia recognition methodologies is disclosed in the article “Onset and Stability for Ventricular Tachyarrhythmia Detection in an Implantable Pacer-Cardioverter-Defibrillator” by Olson et al., published in Computers in Cardiology, Oct. 7-10, 1986, IEEE Computer Society Press, pages 167-170, also incorporated by reference herein in its entirety. Atrial fibrillation detection methodologies are disclosed in Published PCT Application Ser. No. US92/02829, Publication No. WO92/18198, by Adams et al., and in the article “Automatic Tachycardia Recognition”, by Arzbaecher et al., published in PACE, May-June, 1984, pp. 541-547, as well as U.S. Pat. No. 5,247,930, issued to Begemann et al., all of which are incorporated by reference herein in their respective entireties.

In the event an atrial or ventricular tachyarrhythmia is detected and an anti-tachyarrhythmia pacing regimen is desired, appropriate timing intervals for controlling generation of anti-tachyarrhythmia pacing therapies are loaded from microprocessor 51 into the pacer timing and control circuitry 63, to control the operation of the escape interval counters therein and to define refractory periods during which detection of R-waves and P-waves is ineffective to restart the escape interval counters.

Alternatively, circuitry for controlling the timing and generation of anti-tachycardia pacing pulses as described in U.S. Pat. No. 4,577,633, issued to Berkovits et al. on Mar. 25,1986, U.S. Pat. No. 4,880,005, issued to Pless et al. on Nov. 14,1989, U.S. Pat. No. 4,726,380, issued to Vollmann et al. on Feb. 23, 1988 and U.S. Pat. No. 4,587,970, issued to Holley et al. on May 13, 1986, all of which are incorporated herein by reference in their entireties, may also be employed.

In the event that generation of a cardioversion or defibrillation pulse is required, microprocessor 51 may employ an escape interval counter to control timing of such cardioversion and defibrillation pulses, as well as associated refractory periods. In response to the detection of atrial or ventricular fibrillation or tachyarrhythmia requiring a cardioversion pulse, microprocessor 51 activates cardioversion/defibrillation control circuitry 29, which initiates charging of the high voltage capacitors 33 and 35 via charging circuit 69, under the control of high voltage charging control line 71. The voltage on the high voltage capacitors is monitored via VCAP line 73, which is passed through multiplexer 55 and in response to reaching a predetermined value set by microprocessor 51, results in generation of a logic signal on Cap Full (CF) line 77 to terminate charging. Thereafter, timing of the delivery of the defibrillation or cardioversion pulse is controlled by pacer timing/control circuitry 63. Following delivery of the fibrillation or tachycardia therapy microprocessor 51 returns the device to q cardiac pacing mode and awaits the next successive interrupt due to pacing or the occurrence of a sensed atrial or ventricular depolarization.

Several embodiments of appropriate systems for the delivery and synchronization of ventricular cardioversion and defibrillation pulses and for controlling the timing functions related to them are disclosed in U.S. Pat. No. 5,188,105 to Keimel, U.S. Pat. No. 5,269,298 to Adams et al. and U.S. Pat. No. 4,316,472 to Mirowski et al., hereby incorporated by reference herein, each in its respective entirety. Any known cardioversion or defibrillation pulse control circuitry is believed to be usable in conjunction with various embodiments of the present invention, however. For example, circuitry controlling the timing and generation of cardioversion and defibrillation pulses such as that disclosed in U.S. Pat. No. 4,384,585 to Zipes, U.S. Pat. No. 4,949,719 to Pless et al., or U.S. Pat. No. 4,375,817 to Engle et al., all hereby incorporated by reference herein in their entireties, may also be employed.

Continuing to refer to FIG. 5, delivery of cardioversion or defibrillation pulses is accomplished by output circuit 27 under the control of control circuitry 29 via control bus 31. Output circuit 27 determines whether a monophasic or biphasic pulse is delivered, the polarity of the electrodes and which electrodes are involved in delivery of the pulse. Output circuit 27 also includes high voltage switches which control whether electrodes are coupled together during delivery of the pulse. Alternatively, electrodes intended to be coupled together during the pulse may simply be permanently coupled to one another, either exterior to or interior of the device housing, and polarity may similarly be pre-set, as in current implantable defibrillators. An example of output circuitry for delivery of biphasic pulse regimens to multiple electrode systems may be found in the above cited patent issued to Mehra and in U.S. Pat. No. 4,727,877, hereby incorporated by reference herein in its entirety.

An example of circuitry which may be used to control delivery of monophasic pulses is disclosed in U.S. Pat. No. 5,163,427 to Keimel, also incorporated by reference herein in its entirety. Output control circuitry similar to that disclosed in U.S. Pat. No. 4,953,551 to Mehra et al. or U.S. Pat. No. 4,800,883 to Winstrom, both incorporated by reference herein in their entireties, may also be used in conjunction with various embodiments of the present invention to deliver biphasic pulses.

Alternatively, IMD 10 may be an implantable nerve stimulator or muscle stimulator such as that disclosed in U.S. Pat. No. 5,199,428 to Obel et al., U.S. Pat. No. 5,207,218 to Carpentier et al. or U.S. Pat. No. 5,330,507 to Schwartz, or an implantable monitoring device such as that disclosed in U.S. Pat. No. 5,331,966 issued to Bennet et al., all of which are hereby incorporated by reference herein, each in its respective entirety. The present invention is believed to find wide application to any form of implantable electrical device for use in conjunction with electrical leads.

The present invention comprises a method and system for preventing the recurrence of an atrial fibrillation by IMD 10. Generally speaking, an atrial fibrillation is detected. A set of pacing pulses to increase a rate of heart 8 to an intervention rate is transmitted to heart 8 upon the completion of the atrial fibrillation. Finally, the intervention rate is maintained for a predetermined period of time.

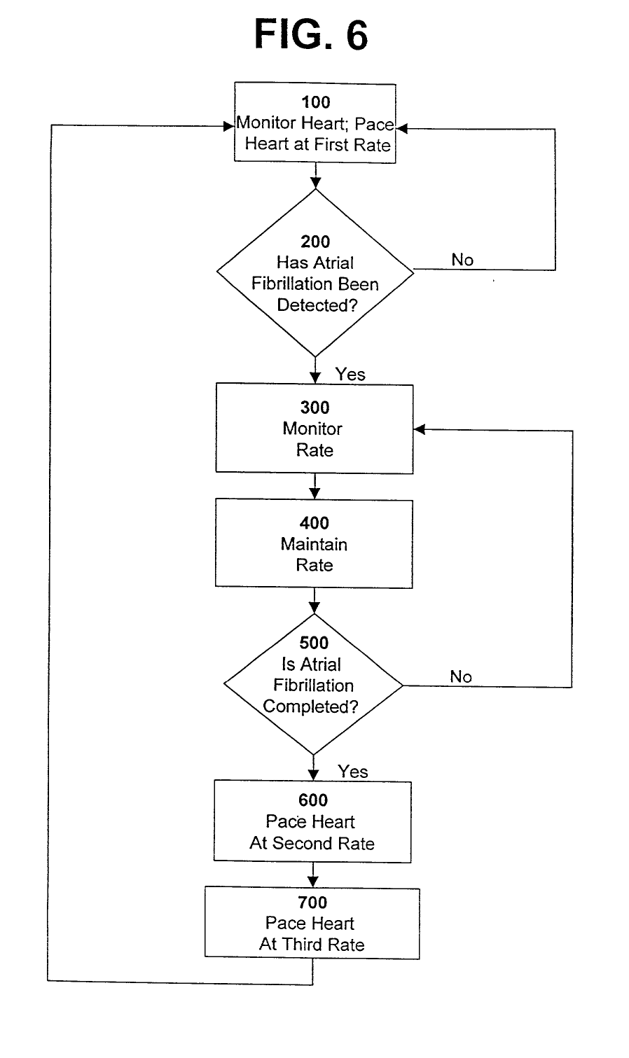

FIG. 6 illustrates one embodiment of a routine for a method and system for preventing the recurrence of an atrial fibrillation in IMD 10. The method for preventing the recurrence of an atrial fibrillation in IMD 10 may be preferably performed by means of a computer algorithm program and/or software, which may be stored integral with, or remote from, IMD 10. Alternatively, the method described herein may be performed in any other similar manner.

The computer algorithm program and/or software may preferably be any program capable of being stored on an electronic medium, such as, for example, RAM 68 or ROM 70, and permitted to be accessed (and consequently run) by microprocessor 64. Alternatively, the method may be performed manually by a programmer electronically programming instructions to IMD 10, either remotely from a location away from IMD 10, or via an electronic connection with IMD 10.

The operation of the software of the present invention may be best explained by reference to three states of operation of the software. The software of the present invention initially operates in a normal programmed state (i.e., DDD(R)). In this state, escape rate timing, tracking and other sensing takes place normally. That is, microprocessor 64, through the software, monitors the heart rate of the patient in a standard manner, in accordance with the mode (i.e., DDD(R)). Upon the detection of an atrial tachyarrhythmia, the software of the present invention “switches” to atrial tachyarrhythmia state.

Once in the atrial tachyarrhythmia state, microprocessor 64, through the software, monitors the heart rate of the patient, and compares the monitored rate with a minimum, or escape rate. In the atrial tachyarrhythmia state, the escape rate may be based on any of the following parameters, all known in the art: lower rate pacing; ventricular rate smoothing, especially if the patient has spontaneous atrial-ventricular conduction; a tachyarrhythmia fallback rate or a rate response rate. The software operating on microprocessor 64 maintains the highest of these rates as the escape rate until the end of the atrial tachyarrhythmia is detected. At this point, the software “switches” to the prevention state.

In the prevention state, microprocessor 64, through the software, initially maintains the escape rate from the previous state. However, the escape rate is then increased to an intervention rate. The intervention rate may be reached in any of the following ways: in one step; according to a programmable incremental level (i.e., in multiple steps); or in one step, arriving at an intermediate rate, followed by a programmable incremental level. To reduce the chance that additional atrial tachyarrhythmias may result, the intervention rate is maintained for a predetermined period of time. After such time, the escape rate is reduced to a lower rate. Upon reaching the lower rate, the software “returns” to the normal state.

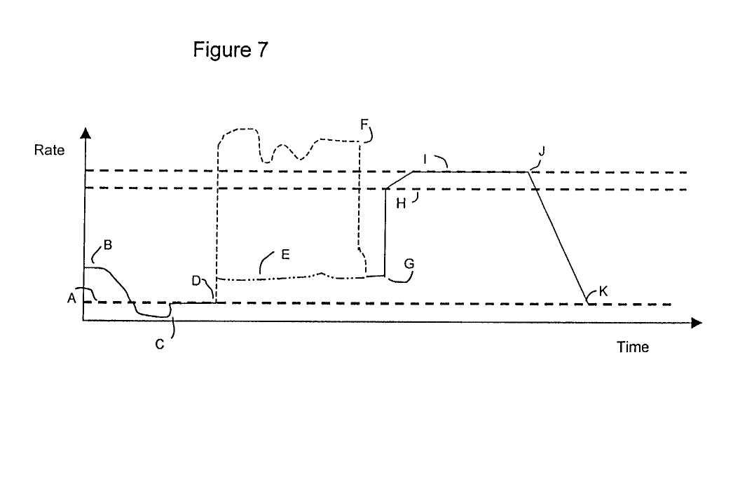

Additional reference to the algorithm of the present invention may be made to FIG. 7, which graphically illustrates the embodiment of the algorithm discussed with reference to FIG. 6.

Returning to FIG. 6, in Block 100, computer algorithm software operating on microprocessor 64 of IMD 10 monitors the heart rate of heart 8. Monitoring the heart rate ensures that heart 8 is being paced (either naturally or through artificial means) at at least the minimum, i.e., escape, rate. This escape rate is shown in FIG. 7 at reference point A, while the actual beat of heart 8 is shown at reference point B. The software may direct IMD 10 to pace heart 8 only upon breach of the escape rate. That is, if the heart rate of heart 8 is greater than the escape rate, the pacing of heart 8 by IMD 10 is inhibited. If, however, the heart rate of heart 8 falls below the escape rate, through direction from the software, IMD 10 “steps in” and transmits a pacing rate at least equal to the escape rate, thus ensuring that heart 8 maintains a healthy rate for the patient. An example of this instance is shown in FIG. 7 at reference point C.

As mentioned above with reference to FIG. 3, monitoring the heart rate of heart 8 may be effected by receiving heart rate signals from the atrium and/or ventricle of heart 8 via sensing circuitry, including pacing and sensing lead 16, 18, EGM amplifier 94, sense amplifier 88, peak sense and threshold measurement unit 90 and comparator/threshold detector 92. Also, as mentioned above with reference to FIG. 3, the pacing of heart 8 by IMD 10 occurs through

In the prevention state, microprocessor 64, through the software, initially maintains the escape rate from the previous state. However, the escape rate is then increased to an intervention rate. The intervention rate may be reached in any of the following ways: in one step; according to a programmable incremental level (i.e., in multiple steps); or in one step, arriving at an intermediate rate, followed by a programmable incremental level. To reduce the chance that additional atrial tachyarrhythmias may result, the intervention rate is maintained for a predetermined period of time. After such time, the escape rate is reduced to a lower rate. Upon reaching the lower rate, the software “returns” to the normal state.

Additional reference to the algorithm of the present invention may be made to FIG. 7, which graphically illustrates the embodiment of the algorithm discussed with reference to FIG. 6.

Returning to FIG. 6, in Block 100, computer algorithm software operating on microprocessor 64 of IMD 10 monitors the heart rate of heart 8. Monitoring the heart rate ensures that heart 8 is being paced (either naturally or through artificial means) at at least the minimum, i.e., escape, rate. This escape rate is shown in FIG. 7 at reference point A, while the actual beat of heart 8 is shown at reference point B. The software may direct IMD 10 to pace heart 8 only upon breach of the escape rate. That is, if the heart rate of heart 8 is greater than the escape rate, the pacing of heart 8 by IMD 10 is inhibited. If, however, the heart rate of heart 8 falls below the escape rate, through direction from the software, IMD 10 “steps in” and transmits a pacing rate at least equal to the escape rate, thus ensuring that heart 8 maintains a healthy rate for the patient. An example of this instance is shown in FIG. 7 at reference point C.

As mentioned above with reference to FIG. 3, monitoring the heart rate of heart 8 may be effected by receiving heart rate signals from the atrium and/or ventricle of heart 8 via sensing circuitry, including pacing and sensing lead 16, 18, EGM amplifier 94, sense amplifier 88, peak sense and threshold measurement unit 90 and comparator/threshold detector 92. Also, as mentioned above with reference to FIG. 3, the pacing of heart 8 by IMD 10 occurs through the process of transmitting a first set of stimulus pulses to heart 8 from digital controller/timer circuitry 74. Output pulse generator 96 receives the pacing stimuli from digital controller/timer circuitry 74, and outputs the pacing stimuli to heart 8 via lead 18.

In Block 200, computer algorithm software operating on microprocessor 64 of IMD 10 determines whether an atrial fibrillation has been detected. An atrial fibrillation occurs when an atrial contraction occurs in accordance with a specific pattern. The specific pattern of atrial contraction may be a pattern faster than the normal pattern of atrial contraction, an irregular atrial contraction pattern, an atrial contraction pattern with a certain morphology, etc. When the atrial fibrillation has been detected, a signal is sent from pacing and sensing lead 16, 18 to microprocessor 64, notifying microprocessor 64 of the detection.

Atrial fibrillations are problematic for a number of factors. First, atrial fibrillations may cause heart 8 to beat at an extremely rapid and/or irregular ventricular rate. Second, atrial fibrillations may reduce the cardiac output of heart 8 due to the loss of the atrial contribution to the ventricular filling (i.e., the loss of the atrial “kick”). Third, atrial fibrillations may reduce the cardiac output of heart 8 due to the rapid and/or irregular ventricular rate. Fourth, the risk of stroke due to thrombus-forming in the fibrillating atria (i.e., blood clotting) may increase with an atrial fibrillation. Fifth, atrial fibrillations may lead to an increased incidence of additional atrial fibrillations. Additionally, the occurrence of any of the above factors may also lead to patient symptoms, which is another problematic feature of atrial fibrillations.

Determination of an atrial fibrillation may be sensed by any of the sensing means, described above with respect to the detection of atrial or ventricular tachyarrhythmias. Additionally, detection of an atrial fibrillation may be confirmed by detecting a sustained series of short and irregular R-R intervals or P-P intervals of an average rate indicative of an atrial fibrillation.

Additionally, detection of an atrial fibrillation may occur when the rate of atrial senses (i.e., the reciprocal value of an AA interval) rises above a predetermined detection rate for a specified number of beats, a percentage of a specified number of beats (i.e., 90% of the total beats) or a specified period of time. Preferably, a programmer may program the predetermined detection rate, the specified number of beats, the percentage of a specified number of beats and the specified period of time into microprocessor 64. Additionally, the detection rate may be variable and dependent upon the patient, physician directives or any other similar factors.

Additionally, detection of an atrial fibrillation may occur when the morphology of the atrial signals indicates an atrial arrhythmia for a specified number of beats, a percentage of a specified number of beats or a specified period of time. Preferably, as is the case above, the programmer may program the specified number of beats, the percentage of a specified number of beats and the specified period of time into microprocessor 64.

Detection of an atrial fibrillation may occur when a known mode-switch (i.e., a switch in the operating (i.e., DDD to DDIR) mode during an atrial tachyarrhythmia) is present for a specified number of beats, a percentage of a specified number of beats or a specified period of time. Preferably, as is the case above, the programmer may program the specified number of beats, the percentage of a specified number of beats and the specified period of time into microprocessor 64.

Detection of an atrial fibrillation may also occur when the beat-to-beat variability of the heart rate of heart 8 exceeds a predetermined beats per minute (bpm) ratio or a predetermined period of time for a specified number of beats, a percentage of a specified number of beats or a specified period of time. The programmer may program the predetermined bpm ratio and the predetermined period of time into microprocessor 64. Furthermore, as is the case above, the programmer may program specified number of beats, the percentage of a specified number of beats and the specified period of time into microprocessor 64.

In FIG. 7, at reference point D, an atrial fibrillation is shown. Detection of the atrial fibrillation preferably occurs by any of the methods described above. Additionally, at this point, the ventricular rhythm of heart 8 may be spontaneous, stabilized by any known means of ventricular rate smoothing or proximate to the lower (or sensor) rate. In FIG. 7, the ventricular rhythm is shown, at reference point E, as being in a stabilized mode.

Upon the detection of an atrial fibrillation, computer algorithm software operating on microprocessor 64 of IMD 10 (now operating in the atrial tachmarrhythmia state) monitors the current pacing rate of heart 8 (Block 300). Preferably, this monitoring is used to determine the end of the atrial fibrillation. Additionally, computer algorithm software operating on microprocessor 64 of IMD 10 maintains the pacing rate of heart 8 (Block 400). The end of the atrial fibrillation is illustrated in FIG. 7 at reference point F. However, microprocessor 64 doesn't detect the termination of the atrial fibrillation (and take the necessary steps to prevent future atrial fibrillations) until reference point G.

Detection of the end of an atrial fibrillation may occur when the rate of atrial senses (i.e., the reciprocal value of an atrial-atrial interval) falls below the predetermined detection rate for a specified number of beats, a percentage of a specified number of beats or a specified period of time. As stated above with regards to the detection of an atrial fibrillation, the programmer may program the predetermined detection rate, the specified number of beats, the percentage of a specified number of beats and the specified period of time into microprocessor 64. Additionally, the detection rate may be variable and dependent upon the patient, physician directives or any other similar factors.

Additionally, detection of the end of an atrial fibrillation may occur when the morphology of the atrial signals indicates the absence of an atrial arrhythmia for a specified number of beats, a percentage of a specified number of beats or a specified period of time. Preferably, as is the case above, the programmer may program the specified number of beats, the percentage of a specified number of beats and the specified period of time into microprocessor 64.

Detection of the end of an atrial fibrillation may occur when a known mode-switch is absent for a specified number of beats, a percentage of a specified number of beats or a specified period of time. Preferably, as is the case above, the programmer may program the specified number of beats, the percentage of a specified number of beats and the specified period of time into microprocessor 64.

Detection of the end of an atrial fibrillation may also occur when the beat-to-beat variability of the heart rate of heart 8 drops below the predetermined bpm ratio or the predetermined period of time (both described above) for a specified number of beats, a percentage of a specified number of beats or a specified period of time. The programmer may program the predetermined bpm ratio and the predetermined period of time into microprocessor 64. Also, as is the case above, the programmer may program the specified number of beats, the percentage of a specified number of beats and the specified period of time into microprocessor 64.

Upon the detection of the completion of an atrial fibrillation (Block 500), computer algorithm software operating on microprocessor 64 of IMD 10 (now operating in the prevention state) paces the atrium of heart 8 with a second set of stimulus pulses (Block 600), in the manner described above. If the completion of an atrial fibrillation in not detected, the software continues the monitoring process described above with regards to Blocks 300 and 400. Preferably, the second set of stimulus pulses paces heart 8 at a rate higher than the first heart rate. This elevated rate, i.e., the intervention rate, is shown in the graphical example of FIG. 7 at reference point 1, where microprocessor 64 directs the increment of the escape rate to the intervention rate, as shown. Also shown in FIG. 7 is the example, described above, in which the elevated rate is increased to the intermediate rate, shown at reference point H, prior to increasing the rate to the intervention rate. Additionally, the second set of stimulus pulses paces heart 8 for a predetermined period of time, as shown by numeral 8. This predetermined period of time may be programmed into microprocessor 64.

The second set of stimulus pulses may be transmitted to heart 8 according to any number of rates. Although the second set of stimulus pulses paces heart 8 at a rate higher than the heart rate monitored in Block 100, there are a number of ways to arrive at the specific pacing rate for which to pace heart 8 at this stage. First, the second set of stimulus pulses may be a simple preset value, programmed into microprocessor 64 of IMD 10 to be a relatively high rate, such as, for example, 120 beats per minute. In any event, this rate, however, should be lower than the ventricular rate during the atrial fibrillation. Second, the second set of stimulus pulses may be a function of the duration and/or the average atrial rate of the recently completed atrial fibrillation.

The second set of stimulus pulses may be a function of the average ventricular rate during the recently-completed atrial fibrillation. The second set of stimulus pulses can be initiated and increased to a rate to prevent or reduce patient symptoms after the end of the atrial fibrillation. If the average ventricular rate is high (as compared with the heart rate discussed with respect to Block 100), the second set of stimulus pulses may be transmitted to heart 8 at a high rate. This is because such a high rate would not aggravate any possible atrial fibrillation symptoms. Alternatively, if the average ventricular rate is low (which may be possibly due to either an atrio-ventricular block or a His-bundle ablation or AV-node of heart 8), the second set of stimulus pulses may be transmitted to heart 8 at a low rate.

Finally, computer algorithm software operating on microprocessor 64 of IMD 10, in Block 700, paces heart 8 with a third set of stimulus pulses, in the manner described above. As shown in FIG. 7, the third set of stimulus pulses is initiated at reference point J and is gradually decreased to the escape rate at reference point K. Preferably, the third set of stimulus pulses makes this transition slowly, gradually decreasing the pacing rate of heart 8, from the level equal to the second set of stimulus pulses towards the escape rate, in an effort to reach the escape rate. Once the heart rate reaches the escape rate, the computer algorithm operating on microprocessor 64 cycles back to Block 100, monitoring heart 8 for another possible atrial fibrillation.

The preceding specific embodiments are illustrative of the practice of the invention. It is to be understood, therefore, that other expedients known to those skilled in the art or disclosed herein, may be employed without departing from the invention or the scope of the appended claims. For example, the present invention is not limited to a method for detecting an atrial fibrillation. The present invention is also not limited to the increase of a heart rate of a mammlian heart, per se, but may find further application as a monitoring means. The present invention further includes within its scope methods of making and using the means described hereinabove.

In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

We claim:

1. A method for preventing the recurrence of an atrial fibrillation of a heart, comprising:
detecting an atrial fibrillation;
transmitting a set of pacing pulses to increase a rate of the heart to an intervention rate upon completion of the atrial fibrillation; and
maintaining the intervention rate for a predetermined period of time.

2. The method of claim 1, further comprising, prior to detecting the atrial fibrillation:
monitoring the rate of the heart; and
transmitting a set of pacing pulses to increase the rate of the heart if the rate of the heart is lower than an escape rate.

3. The method of claim 2, further comprising maintaining the set of pacing pulses which increases the rate of the heart if the rate of the heart is lower than the escape rate until the completion of the atrial fibrillation.

4. The method of claim 2, wherein the intervention rate is greater than the escape rate.

5. The method of claim 1, further comprising transmitting a set of pacing pulses to decrease the rate of the heart to an escape rate after the predetermined period of time.

6. The method of claim 5, wherein the set of pacing pulses which decreases the rate of the heart to the escape rate initially paces the heart at a rate equal to the intervention rate.

7. The method of claim 6, wherein the set of pacing pulses which decreases the rate of the heart to the escape rate gradually decreases the rate of the heart.

8. The method of claim 1, further comprising monitoring the rate of the heart after detecting the atrial fibrillation.

9. The method of claim 1, wherein the set of pacing pulses which increases the rate of the heart to the intervention rate paces the heart at an intermediate rate prior to pacing the heart at the intervention rate.

10. The method of claim 9, wherein the intervention rate is greater than the intermediate rate.

11. The method of claim 1, wherein the intervention rate is based on a length of the atrial fibrillation.

12. The method of claim 1, further comprising monitoring an atrial fibrillation rate.

13. The method of claim 12, wherein the intervention rate is based on the atrial fibrillation rate.

14. The method of claim 1, wherein the intervention rate is based on an average ventricular rate of the heart.

15. The method of claim 1, wherein the heart is a human heart.

16. An implantable medical device comprising:
a processor;
a controller operably connected to the processor;
at least one pacing lead operably connected to the controller; and
at least one sensing lead operably connected to the controller; wherein
the sensing lead senses an atrial fibrillation and sends a signal to the processor, which instructs the controller to increase a heart rate of a heart by transmitting a set of pacing pulses via the pacing leads to the heart upon the completion of the atrial fibrillation.

17. The implantable medical device of claim 16, wherein the heart rate is monitored after sensing the atrial fibrillation.

18. The implantable medical device of claim 16, wherein, prior to the atrial fibrillation, the processor instructs the controller to pace the heart at a rate equal to an escape rate if the heart rate falls below the escape rate.

19. The implantable medical device of claim 16, wherein the increased heart rate is maintained until the completion of the atrial fibrillation.

20. The implantable medical device of claim 16, wherein the increased heart rate is maintained for a predetermined period of time after the completion of the atrial fibrillation.

21. The implantable medical device of claim 16, wherein the set of pacing pulses paces the heart at a rate at least as great as an escape rate.

22. The implantable medical device of claim 16, wherein the set of pacing pulses paces the heart at an intervention rate.

23. The implantable medical device of claim 16, wherein the set of pacing pulses paces the heart at a rate which is based on a length of the atrial fibrillation.

24. The implantable medical device of claim 16, further comprising monitoring an atrial fibrillation rate.

25. The implantable medical device of claim 24, wherein the set of pacing pulses paces the heart at a rate which is based on the atrial fibrillation rate.

26. The implantable medical device of claim 16, wherein the set of pacing pulses paces the heart at a rate which is based on an average ventricular rate.

27. The implantable medical device of claim 16, wherein:
the heart rate is increased by the set of pacing pulses followed by a set of second pacing pulses; and
the set of second pacing pulses initially paces the heart at a rate equal to the set of pacing pulses.

28. The implantable medical device of claim 27, wherein the set of second pacing pulses paces gradually reduces the heart rate of the heart.

29. The implantable medical device of claim 28, wherein the set of second pacing pulses eventually paces the heart at the escape rate.

30. The implantable medical device of claim 16, wherein the heart is a human heart.

31. An implantable medical device system for preventing the recurrence of an atrial fibrillation of a heart, comprising:
means for detecting an atrial fibrillation;
means for transmitting a set of pacing pulses to increase a rate of the heart to an intervention rate upon completion of the atrial fibrillation; and
means for maintaining the intervention rate for a predetermined period of time.

32. The implantable medical device system of claim 31, further comprising, prior to detecting the atrial fibrillation:
means for monitoring the rate of the heart; and
means for transmitting a set of pacing pulses to increase the rate of the heart if the rate of the heart is lower than an escape rate.

33. The implantable medical device system of claim 32, further comprising means for maintaining the set of pacing pulses which increases the rate of the heart if the rate of the heart is lower than the escape rate until the completion of the atrial fibrillation.

34. The implantable medical device system of claim 32, wherein the intervention rate is greater than the escape rate.

35. The implantable medical device system of claim 31, further comprising means for transmitting a set of pacing pulses to decrease the rate of the heart to an escape rate after the predetermined period of time.

36. The implantable medical device system of claim 35, wherein the set of pacing pulses which decreases the rate of the heart to the escape rate initially paces the heart at a rate equal to the intervention rate.

37. The implantable medical device system of claim 36, wherein the set of pacing pulses which decreases the rate of the heart to the escape rate gradually decreases the rate of the heart.

38. The implantable medical device system of claim 31, further comprising means for monitoring the rate of the heart after detecting the atrial fibrillation.

39. The implantable medical device system of claim 31, wherein the set of pacing pulses which increases the rate of the heart to the intervention rate paces the heart at an intermediate rate prior to pacing the heart at the intervention rate.

40. The implantable medical device system of claim 39, wherein the intervention rate is greater than the intermediate rate.

41. The implantable medical device system of claim 31, wherein the intervention rate is based on a length of the atrial fibrillation.

42. The implantable medical device system of claim 31, further comprising means for monitoring an atrial fibrillation rate.

43. The implantable medical device system of claim 42, wherein the intervention rate is based on the atrial fibrillation rate.

44. The implantable medical device system of claim 31, wherein the intervention rate is based on an average ventricular rate of the heart.

45. The implantable medical device system of claim 31, wherein the heart is a human heart.

46. A computer usable medium for storing a program for preventing the recurrence of an atrial fibrillation of a heart comprising:
computer readable program code that detects an atrial fibrillation;
computer readable program code that transmits a set of pacing pulses to increase a rate of the heart to an intervention rate upon completion of the atrial fibrillation; and
computer readable program code that maintains the intervention rate for a predetermined period of time.

47. The program of claim 46, further comprising, prior to detecting the atrial fibrillation:
computer readable program code that monitors the rate of the heart; and
computer readable program code that transmits a set of pacing pulses to increase the rate of the heart if the rate of the heart is lower than an escape rate.

48. The program of claim 47, further comprising computer readable program code that maintains the set of pacing pulses which increases the rate of the heart if the rate of the heart is lower than the escape rate until the completion of the atrial fibrillation.

49. The program of claim 47, wherein the intervention rate is greater than the escape rate.

50. The program of claim 46, further comprising computer readable program code that transmits a set of pacing pulses to decrease the rate of the heart to an escape rate after the predetermined period of time.

51. The program of claim 50, wherein the set of pacing pulses which decreases the rate of the heart to the escape rate initially paces the heart at a rate equal to the intervention rate.

52. The program of claim 51, wherein the set of pacing pulses which decreases the rate of the heart to the escape rate gradually decreases the rate of the heart.

53. The program of claim 46, further comprising computer readable program code that monitors the rate of the heart after detecting the atrial fibrillation.

54. The program of claim 46, wherein the set of pacing pulses which increases the rate of the heart to the intervention rate paces the heart at an intermediate rate prior to pacing the heart at the intervention rate.

55. The program of claim 54, wherein the intervention rate is greater than the intermediate rate.

56. The program of claim 46, wherein the intervention rate is based on a length of the atrial fibrillation.

57. The program of claim 46, further comprising computer readable program code that monitors an atrial fibrillation rate.

58. The program of claim 57, wherein the intervention rate is based on the atrial fibrillation rate.

59. The program of claim 46, wherein the intervention rate is based on an average ventricular rate of the heart.

60. The program of claim 46, wherein the heart is a human heart.
